(19) United States
(12) Patent Application Publication
(10) Pub. No.: US 2023/0256416 A1
(43) Pub. Date: Aug. 17, 2023

(54) METHOD FOR PRODUCING A MIXED OXIDE CARRIER AND FURTHER FINISHING THEREOF INTO A CATALYST FOR PRODUCING ALKYL METHACRYLATES

(71) Applicant: Röhm GmbH, Darmstadt (DE)

(72) Inventors: Steffen Krill, Muehltal (DE); Andreas Rühling, Darmstadt (DE); Andreas Tepperis, Bad Koenig (DE)

(73) Assignee: Röhm GmbH, Darmstadt (DE)

(21) Appl. No.: 18/006,209

(22) PCT Filed: Jul. 1, 2021
(86) PCT No.: PCT/EP2021/068113

§ 371 Date: Jan. 20, 2023

(30) Foreign Application Priority Data
Jul. 24, 2020 (EP) 20187639.8

(51) Int. Cl. B01J 21/12; B01J 35/10; B01J 37/02; C07C 67/39; B01J 23/89

(52) U.S. Cl. CPC B01J 21/12; B01J 23/8946; B01J 35/1019; B01J 35/1047; B01J 35/1061; B01J 37/0201; C07C 67/39

(57) ABSTRACT

A new method can be used for producing suitable improved carrier materials as a base material for catalysts for carrying out a direct oxidative esterification. In general, the catalyst is used to convert aldehydes with alcohols in the presence of oxygenic gases directly to the corresponding ester, for example, where (meth)acrolein can be converted to methyl(meth)acrylate. The catalysts used are characterized in particular by high mechanical and chemical stability as well as by good catalytic performance even over very long periods of time. This applies in particular to an improvement of catalyst service life, activity and selectivity in comparison to other catalysts.

### FIELD OF THE INVENTION

The present invention relates to a novel process for preparing suitable improved support materials as base material for catalysts for performance of a direct oxidative esterification. In general, the catalyst serves for reaction of aldehydes with alcohols in the presence of oxygenous gases directly to give the corresponding ester, by means of which, for example, (meth)acrolein can be converted to methyl (meth)acrylate.

The catalysts used for this purpose in accordance with the invention are especially notable for high mechanical and chemical stability and for good catalytic performance even over very long periods. This relates more particularly to an improvement in catalyst service life, activity and selectivity over prior art catalysts.

What is special about this novel catalyst of the invention is that the catalytic performance thereof can be distinctly enhanced by the drying time and storage time between the process steps up to calcination. In the use of the catalysts of the invention, the activity of the reaction is much more stable over a long period of operation.

The parent mixed oxide support material and the resulting catalyst based on silicon dioxide, aluminum oxide and magnesium oxide additionally has, by means of classification, a grain size distribution in which the fines content is greatly reduced, as a result of which, as an important aspect, it is possible to suppress and significantly reduce the formation of by-products that boil close to the desired ester, especially methyl methacrylate, or form azeotropes with the product or the reactants that are difficult to separate.

The novel catalyst type thus allows production of MMA purities and qualities that are much higher than with the catalysts described to date in the prior art.

### PRIOR ART

The catalytic oxidative esterification of aldehydes for preparation of carboxylic esters is described extensively in the prior art.

For example, it is possible in this way to produce methyl methacrylate very efficiently from methacrolein (MAL) and methanol. US 5,969,178 and US 7,012,039 in particular describe a process for continuously preparing MMA from isobutene or tert-butanol. This process has the following steps: 1) oxidation of isobutene or tert-butanol to methacrolein and 2) direct oxidative esterification of MAL with methanol to give MMA with a Pd-Pb catalyst on an oxidic support. Even though conversion and selectivity are high in principle, it is necessary to constantly supply a lead component for continuous operation since the catalyst has a continuous low loss of lead ions. The workup and removal of lead-containing wastewaters entail great technical complexity and ultimately also cause critical heavy metal-containing waste materials and wastewaters that have to be treated separately, at high ongoing cost.

EP 2210664 discloses a catalyst having, in the outer region, in the form of what is called an eggshell structure, nickel oxide and gold nanoparticles on a support composed of silicon dioxide, aluminum oxide and a basic element, especially an alkali metal or alkaline earth metal. The nickel oxide is enriched at the surface, but is also present in lower concentrations in deeper layers of the catalyst particle. Such a catalyst exhibits very good activities and selectivities. However, the catalyst produced by the inventive preparation method from this application is relatively sensitive to abrasion and unstable. In the experimental use of these above-described catalysts, in addition, there is relatively high contamination with methyl isobutyrate, the formal hydrogenation product of MMA, which increases separation complexity and energy expenditure in product isolation.

The particular preparation method for production of the eggshell structure and the use of not uncritical nickel salts in the production of the catalyst place particular demands on industrial apparatus and the handling of fine nickel-containing dusts as inevitably occur in catalyst manufacture, for example in the process step of drying and calcining. Here too, the nickel doping component is described as necessary alongside the gold nanoparticles and the particular anisotropic, inhomogeneous distribution of gold and dopant in order to achieve high activity and selectivity over a long period of time.

EP 3244996 discloses a similar catalyst system, wherein a doping element used in place of nickel oxide is cobalt oxide as a component alongside gold. Here too, a mixed oxide support is used, with achievement of better results overall than in EP 2210664, where the hydrogenated MMA by-product methyl isobutyrate is formed in small traces here too. There is no discussion of the effect of the grain size spectrum of the catalyst on production of by-products, or the problem of removal of catalyst fines in a continuous reaction regime. There is no description in this or other publications of important manufacturing steps and manufacturing conditions that have a considerable effect on catalyst performance under reaction conditions.

In patent specifications US 7,326,806, US 2013/0172599 and US 9,480,973, the composition of the mixed oxide support and the influence that it has on hydrolysis stability and abrasion are examined and optimized, but only experiments on the laboratory scale are reported here. The parameters for spray drying, calcination and classification that are important on an industrial and laboratory scale and the ultimately resultant grain size distribution are not a topic of discussion. As is known to the person skilled in the art, grain size distribution, especially the fines content of a catalyst, plays a major role in the choice of reactor and filtration, in order to prevent loss of catalyst and hence the metallic active component in operation. The contamination of process wastewater with metals means that technical measures have to be taken for protection of man and the environment, which incur high costs together with any loss of precious metals.

Patent specification US RE38,283 describes and discusses the original composition of the mixed oxide support which is taken up in the abovementioned documents and adapted, by means of which good hydrolysis stability and stability toward organic acids is achieved. However, the same aspects are missing here as in the above-cited specifications.

Moreover, all the systems, processes and catalysts described in the prior art do not describe, or only insufficiently describe, the formation of critical by-products, especially including hydrogenated by-products, which play an essential role in the isolation of market-standard qualities of alkyl methacrylates, especially MMA. Such a critical by-product which can be separated from MMA only with considerable apparatus complexity and considerable use of energy is methyl isobutyrate, also referred to as methyl isobutyrate. This by-product is the corresponding saturated hydrocarbon of an unsaturated compound prepared in the process, for example the hydrogenated conversion product of the alkyl methacrylate, and has a very similar boiling point, which makes distillative removal very complex and in some cases possible only with loss of yield. This component occurs in very many standard industrial methyl methacrylate processes and is ultimately also found in the commercial MMA product as used, for example, for the production of PMMA, but in concentrations much less than 500 ppm, typically also less than 100 ppm.

What is common to all catalysts and mixed oxide support systems described in the prior art is that these, in the course of production, undergo one or more drying steps and at least one calcination, with removal of the water used in the reaction and any salts, e.g. nitrates or acetates. While the literature here is restricted to laboratory examples, the parameter of drying time and the associated residual moisture content are consequently not examined and reported in a differentiated manner. However, Ma et. al. in “Heterogeneous gold catalysts and catalysis” reports that catalysts with gold as precious metal component are frequently subject to the problem of sintering, particularly in the moist state, which greatly reduces catalyst activity on account of rising nanoparticle diameter. Thus, specifically the process steps of drying and calcination, in terms of their duration and the time intervals for application of the active (precious) metal component to the support material, are of crucial importance for an active catalyst having long-term stability.

In the reproduction of the processes and catalysts described in the prior art, the water contents of the intermediates, the dwell time of the various individual process steps and especially the storage times between the individual process steps are found to be essential for the reproducible production of a catalyst produced batchwise in component steps, or of the underlying support material. These connections are fundamentally not described in the prior art.

Overall, various catalysts are thus described in the prior art for direct oxidative esterification (DOE), for example for the reaction of unsaturated aldehydes such as acrolein and methacrolein with alcohols to give the respective carboxylic esters.

However, there is only insufficient discussion of the formation of by-products and the influence of an industrially manufactured support material on these by-products and general catalyst management in the course of operation.

### OBJECT

It was a primary object of the present invention to develop a novel process for producing a mixed oxide support and a catalyst based thereon and coated with active metal-containing components on an industrial scale, wherein the resulting catalyst is suitable for the direct oxidative esterification of aldehydes to carboxylic esters. The mixed oxide support itself, and the catalyst produced on the basis of said mixed oxide support, should have high mechanical and chemical stability, produce a lower level of by-products overall compared to the prior art, and should simultaneously be easier to handle in filtration under reaction conditions.

A particular problem addressed was that this process is to be suitable for the oxidative esterification of methacrolein to an alkyl methacrylate, especially to MMA.

A particularly important partial aspect of the objective underlying the present invention is to achieve efficient and reduced use of (precious) metal components, which, preferably in accordance with the invention, are deposited to a greater than proportional degree on the catalyst fines. These catalyst fines lead to increased formation of secondary components. Furthermore, when the catalyst is used in long-term operation, a high proportion of the fines is lost as catalyst discharge, in the simplest case in a filtration.

In this connection, it was a further object of the invention to provide a catalyst material having a significantly reduced tendency to sintering of the precious metal component or leaching. This is true not just of the consumption and use of the catalyst in a direct oxidative esterification of (meth)acrolein, for example.

The suppression of sintering and leaching of metal compounds is especially an explicit object in the manufacture of the silicon oxide-based support material and of the impregnated catalyst based thereon. If this is not sufficiently controlled and is conducted with suitable measures according to the invention, there is partial loss of the desired distribution structure of the active components in the catalyst material, or formation of a less active catalyst.

Especially the observance of storage times and manufacturing times of individual phases and the storage times of the intermediate between the individual phases of production according to a (i) to (iii) and b (i) to b(vi) is crucial for the obtaining of an improved catalyst and thus constitutes a further object of the present invention.

This defines the aim of a stable catalyst activity over the entire lifetime of the catalyst in use.

A particularly important partial aspect of the objective was that of providing a novel process which, especially in the conversion of aldehydes to carboxylic esters, enables reduced formation of by-products and hence higher selectivity. Such a by-product in the case of MMA synthesis, for example, is methyl isobutyrate, the saturated or hydrogenated form of MMA.

Further problems which are not stated explicitly may become apparent from the description, the examples, the claims or the overall context of the present invention.

### SOLUTION

These objects are achieved by the provision of a novel process for producing a support material and a catalyst based on said support material for an oxidative esterification. This novel process has the two component processes of a) production of a support and b) production of a catalyst. In particular, the novel process is characterized by the following aspects of the two component processes a) and b):

In component process a), an oxidic support is produced. The resulting support includes at least one or more than one oxide of at least one or more than one of the following elements: silicon, aluminum, one or more alkaline earth metals, titanium, zirconium, hafnium, vanadium, niobium, tantalum, yttrium and/or lanthanum.

In addition, component process a) comprises the following process steps:
(i) The reaction of one or more compounds selected from silicon compounds, aluminum compounds, alkaline earth metal compounds, titanium compounds, zirconium compounds, hafnium compounds, vanadium compounds, niobium compounds, tantalum compounds, yttrium compounds and/or lanthanum compounds at a temperature T₁ < 100° C. This reaction affords a suspension.
(ii) Spray-drying of the suspension from process step (i) at a temperature T₂ of > 110° C. to obtain a solid-state material. This solid-state material includes 0.1% to 20% by weight of water and 0.1% to 35% by weight of anions of one or more Brønsted acids.
(iii) Calcination of the solid-state material from (ii) at a temperature T₃ between 300 and 800° C. This affords a second solid-state material including 0.01% to 5% by weight of water and 0.01% to 0.5% by weight of anions of a Brønsted acid.
(iv) Optionally, but preferably, the support powder resulting from one of the support steps (i) to (iii), preferably from support step (iii), is subjected to a classifying step.

Component process b) in which a catalyst is produced from the oxidic support material from component process a) especially comprises the following process steps:
(i) Reaction of the support material from a) with a water-soluble precious metal salt.
(ii) Addition of a further soluble metal salt simultaneously or after process step b (i).
(iii) This is followed by the separation of the impregnated support from the mother liquor or the supernatant solution from process step b (ii), with subsequent washing. This affords a washed impregnated support containing 1.0% to 50% by weight of water.
(iv) Drying the impregnated support for 0.1 to 40 h at a temperature T₄ between 30 and 250° C. This affords a dried impregnated support containing only 0.1% to 10% by weight of water. The drying can be effected, for example, in a paddle dryer or tray dryer. The drying can take place batchwise or else continuously.
(v) Calcination of the dried impregnated support from (iv) at a temperature T₅ between 250 and 700° C. and for a residence time between 0.1 and 5 h. A catalyst having a BET surface area of 100 to 300 m²/g with a pore volume of 0.2 to 2.0 mL/g and a pore diameter of 3 to 12 nm is obtained.

Preferred embodiments are specified hereinafter with regard to the individual process steps. These individual preferred features - unless stated otherwise - are implementable separately from one another or together or synchronously, i.e. at the same time. It should be emphasized that not only the process steps listed here as being essential have synergistic effects in the production of the catalysts or support materials; instead, the preferred or optional embodiments can quite possibly also cause further such effects.

With regard to the process steps of component process a), particular mention is made of the following optional and preferred embodiments:
(i) The reaction to obtain a suspension is preferably conducted batchwise. Further preferably, the oxidic support to be produced includes silicon oxide, aluminum oxide and at least one alkaline earth metal oxide, more preferably magnesium oxide. Alternatively or additionally, the support to be produced includes at least 2% by weight of titanium dioxide, where the support may even consist entirely of titanium dioxide. In process step a (i), corresponding nonoxidic compounds are preferably used to obtain these oxides. Usable compounds are those which are especially partly soluble or, under batch conditions, fully soluble, and which form a precipitate together after reaction. The person skilled in the art is aware of these soluble compounds; preference is given to those salts that can be broken down in the calcination step (iii), such that the desired oxides can be formed more or less without residues. Nitrate salts and acetates meet these conditions; many other anions of corresponding Brønsted acids meet these criteria as well.
(ii) The spray drying of suspension from process step (i) is preferably conducted continuously or semicontinuously. In the spray drying, the temperature and volume flow rate of the drying gas are preferably adjusted such that the laden drying gas at the exit from the spray tower is above the condensation temperature of water by 10 to 40° C., more preferably by 20 to 30° C. The suspension obtained in a) (i) can be sprayed with nozzles or atomizers known to the person skilled in the art; particular preference is given to one-phase nozzles and rotary atomizers.
(iii) The calcination of the solid-state material from (ii) is preferably conducted continuous or semicontinuously, more preferably in a rotary tube. Alternatively, the calcination can also be affected batchwise, in which case it is possible to utilize tray dryers or shaft furnaces. More preferably, the time between spray drying and calcination in steps a (ii) and a (iii) is not longer than 5 days. More preferably, the calcination is effected in the presence of an oxygenous gas. In this way, NO₂ formed can be removed more efficiently.
(iv) The performance of the optional classifying step is distinctly preferred. More preferably, the solid-state material from process step a (iii) is treated in such a way that the proportion of particles having a diameter of less than 20 µm is reduced.

Component process b) in which a catalyst is produced from the oxidic support material from component process a) especially comprises the following process steps:
(i) The reacting of the support material from a) with a water-soluble precious metal salt is preferably conducted batchwise. It is especially preferable, in process step b (i), first to produce an aqueous suspension of the oxidic support from a. and to mix it with the water-soluble precious metal salt. It is optionally possible to add the water-soluble precious metal salt as early as during the production of the suspension, but it is preferable to add it to the suspension.
(ii) Additionally or after the addition of the further soluble metal salt, preference is given to additionally adding a basic aqueous solution to the mixture.
(iii) Preference is given to working up the mother liquor separated off in process step b (iii) in such a way that remaining precious metal salts and other metal salts are recovered.
(iv) The impregnated support is preferably dried at an absolute pressure between 0.01 and 5 bar and/or in the presence of an inert drying gas. More preferably, the time between washing and calcination in steps b (iv) and b (v) is not longer than 4 days.
(v) The dried impregnated support from (iv) is optionally calcined batchwise. However, this is preferably effected continuously or semicontinuously in a rotary tube. More preferably, the calcination is effected in the presence of an oxygenous gas. In this way, NO₂ formed can be removed more efficiently. A catalyst having a BET surface area of 180 to 250 m²/g with a pore volume of 0.2 to 0.7 mL/g and a pore diameter of 3 to 9 nm is preferably obtained.

Preferably, the supports and the resulting catalysts have a diameter between 10 and 200 µm. Such catalysts may be used efficiently in a slurry reactor.

The particle size of the support comprising mixed oxides based on silicon dioxide according to features a (i) to a (iii) may be freely chosen and obtained in various orders of size depending on the chosen production process and apparatus used for the purpose. It is possible to adjust the order of size and other physical characteristics, for example BET surface area, pore volume and pore diameter, by variations of parameters in the steps of spray drying and calcination.

In principle, there is a correlation between particle size and spherical and geometric shape and the later use of the catalysts under reaction conditions.

By the process of the invention, it is possible to produce pulverulent supports and catalysts that are then in the form of a suspension in a stirred reactor system and are used in slurry form in the reaction medium. The support and catalyst manufactured therefrom, in terms of dimensions, then attain an order of size of 1 to 300 µm, preference being given in accordance with the invention to reducing and largely eliminating the fines after manufacturing step a (iii) in a classifying step a (iv).

According to the type of classification chosen, the fines fraction and also the coarse fraction of the pulverulent support is thus influenced, said pulverulent support then being used after classification for catalyst manufacture b (i) to b (vi). Preferred methods of influencing grain size are air classification and sieving, and combinations of these methods; the person skilled in the art is aware of further methods in order to achieve said object of delimiting the grain spectrum. The classification adjusts the grain spectrum of the material obtained after spray drying to 10 to 200 µm; these figures are based on the outcome in which more than 95% by weight of the pulverulent material obtained is within this grain band range. Particular preference is given to a classification in step a (iv), the result being that more than 95% by weight of the pulverulent material obtained has a grain spectrum between 20 and 150 µm. The silicon dioxide-based material according to a (i) to (iv), after spray drying and classification, is in spherical or elliptical form. Sphericity here has an average value of greater than 0.85, preferably greater than 0.90 and more preferably greater than 0.93.

Sphericity here is the ratio of the circumference of the circle of equal size to the actual circumference. The result is a value between 0 and 1. The smaller the value, the more irregular the particle shape. This is the consequence of the fact that an irregular particle shape is manifested in an increased circumference. The comparison is in principle made with the circle of equal area, since this has the smallest of all possible circumferences for a projection area.

In an alternative embodiment, however, catalysts are produced for what are called fixed bed reactors by the process of the invention. Such catalysts, or the support materials underlying them, have a much greater diameter, more preferably between 0.1 and 100 mm. Optionally, therefore, for production of such a catalyst, in a process step a (v), a solid-state material from one of process steps a (ii), a (iii) or a (iv), preferably from process step a (iv), is subjected to a shaping step in such a way that a shaped body having a diameter between 0.1 and 100 mm is obtained. When a process step a (v) is effected after one of process steps a (ii) or a (iii), the further process steps a (iii) and a (iv) or only a (iv) are conducted thereafter.

In a further embodiment, the mixed oxide material obtained in a (iii), or the calcined and classified material optionally obtained in a (iv), is subjected to a shaping step.

Specific examples of the shape of the resulting material are spherical, elliptical, tablet-shaped, cylindrical, annular, acicular, hollow-cylindrical, honeycomb-shaped compacts having an order of size with dimensions of 300 µm to several cm. The person skilled in the art knows how such shaping processes are implemented industrially. In the simplest case, the pulverulent material is initially charged in an extruder in paste form with or without processing auxiliaries, and extruded under pressure using a nozzle that determines the shape.

If it is used as catalyst or catalyst support, the shape of the silicon dioxide-based material in the present embodiment may be altered in a suitable manner depending on a reaction system to be used. When the silicon dioxide-based material is used, for example, in a fixed bed reaction, it preferably has the shape of a hollow cylinder or a honeycomb that causes a low pressure drop.

In addition, before, during or after the conversion of the oxidic support in process steps b) (i) and (ii), a water-soluble Brønsted or Lewis acid may be added. This is preferably an aqueous solution of a metal salt having the +II or +III oxidation state, for example aluminum nitrate or iron(III) nitrate. In this way, it is possible to create a thin (outer) protective layer around the shell comprising the active component that further minimizes the loss of precious metal and hence catalyst activity. By addition of an appropriate metal salt, magnesium oxide present in the outer layer of the oxidic support is leached therefrom in an acid-base reaction. The resulting defects may be filled by the added metal salt and likewise converted to an oxidic form in the calcination of the catalyst material, thus maintaining the chemical and physical stability of the support or catalyst; preferably, by choice of the metal salt, chemical and physical stability are increased further, for example to counter abrasion. No precious metal can be deposited at these magnesium oxide-free sites during the catalyst production, which gives rise to a precious metal-free outer layer that functions as an (outer) protective layer for the catalyst. This - as described above - minimizes the loss of precious metal and hence catalyst activity. For avoidance of any lack of clarity, it should be noted that this further metal salt in the form of a Lewis acid is explicitly not the metal salt from process step b) (ii).

In a further preferred embodiment, it is possible that the above-described shell structure that features a very small proportion of precious metal is also achieved by adding a non-metal-containing acidic compound. In the simplest case, this may be an aqueous solution of a Brønsted acid, for instance nitric acid. In this case, the basic alkali metal or alkaline earth metal oxide, for instance magnesium oxide, is measurably (partly) leached out of the shell, but not exchanged for metal ions as in the case described above.

The resulting outer protective layer, caused both in the case of metal salts or Brønsted acids, preferably has a thickness of 0.01 to 10 µm, more preferably of 0.1 to 5 µm, in order to prevent any reduction in catalyst activity as a result of restrictions in mass transfer or through limitation of diffusion of the reactants and products.

The drying time for the impregnated support material, as described in b) (iv), is less than 4 days, preferably less than 2 days and more preferably less than 1 day, where the residual moisture content of the dried support material is less than 5% by weight, preferably less than 3% by weight and more preferably less than 2.5% by weight. A necessary condition to be achieved is that the remaining amount of water in the dried impregnated support material, during the calcination, cannot damage the calcination equipment, for instance a rotary tube, or there cannot be any resultant condensation of the water in the calcination equipment or the offgas system thereof. A shortened drying time brings the advantage that gold, which is only weakly fixed at first, has less time for a sintering process, which is facilitated in the presence of water and salts, for instance chloride or nitrate, especially at elevated temperatures, as customary in a drying operation. This sintering process increases the average particle diameter, which leads to lower catalyst performance.

As well as the process of the invention for production of the catalyst described, another part of the invention is the use thereof for continuous preparation of carboxylic acids from aldehydes and alcohols in the presence of an oxygenous gas in the liquid phase. The catalyst here is suspended heterogeneously in the reaction matrix.

This reaction is preferably effected at a temperature between 20 and 120° C., a pH between 5.5 and 9, and a pressure between 1 and 20 bar. Particular preference is given to conducting this reaction in such a way that the reaction solution contains between 2% and 10% by weight of water.

In an alternative embodiment of the present invention, the use of the catalyst produced in accordance with the invention for continuous preparation of carboxylic acids from aldehydes and alcohols in the presence of an oxygenous gas is effected using the catalyst in a fixed bed.

It should additionally be noted that the catalysts of the invention, as well as direct oxidative esterification, may also be used for other oxidation reactions, for instance the preparation of carboxylic acids from aldehydes in the presence of water and optionally a solvent.

As well as the process of the invention for preparation of a catalyst and the use of that catalyst, another part of the present invention is, in particular, a catalyst producible in accordance with the invention itself.

### EXAMPLES

### Example 1a - Support Production & Spray Drying

An enamel-lined reactor was initially charged with 434 kg of silica sol (Köstrosol 1530, primary particles 15 nm, 30% by weight of SiO₂ in H₂O), which was cooled down to 10° C. with vigorous stirring. The silica sol dispersion was adjusted to a pH of 2 with 60% nitric acid in order to break up the basic stabilization (sodium oxide).

In a second enameled vessel, a mixture of 81.2 kg of aluminum nitrate nonahydrate, 55.6 kg of magnesium nitrate hexahydrate and 108.9 g of demineralized water was made up. The mixture cooled down in the course of dissolution while stirring and had a pH just below 2. After complete dissolution, 3.2 kg of 60% nitric acid was added.

Subsequently, the metal solution was added to the silica sol dispersion in a controlled manner over the course of 30 minutes. On completion of addition, the mixture was heated to 50° C. and the resulting dispersion was gelated for 4 hours, with a pH of 1 at the end. The resultant viscosity was below 10 mPas.

The suspension (solids content about 30% by weight) was pumped at a temperature of 50° C. with a feed rate of 20 kg/h into a pilot spray tower having a diameter of about 1.8 m and sprayed therein by means of an atomizer disk at 10 000 revolutions per minute, giving a spherical material. The drying gas supplied was adjusted at 180° C. such that the emerging cold drying gas had a temperature of 120° C. The resultant white spherical material had a residual moisture content of 10% by weight. The residual moisture content was determined by drying to constant weight at 105° C.

The use of nitrates per kg of material was just below 0.5 kg, which corresponded to a proportion of about 30% by weight in the spray-dried material.

### Example 1b - Calcination

The material spray-dried in example 1a was calcined at 650° C. under air in a rotary tube-like continuous apparatus. The dwell time was adjusted by means of internals and optimizations of the angle of inclination such that the resultant material after calcination had a nitrate content below 1000 ppm. The residual amount of nitrate was determined by means of ion chromatography with a conductivity detector and relates to the amount of soluble nitrate in demineralized water.

At an angle of inclination of 0.5 degree, a dwell time of 45 minutes was established, with quantification of the nitrate content by means of double determination by ion chromatography at 936 ppm. The water content of the material directly after calcination was determined analogously to example 1a and was 1.3% by weight.

The nitrogen oxides released - calculated as NO₂ - were 0.3 kg/kg of material and were collected in a DeNOₓ scrubber.

### Example 1c - Classification

The material obtained from example 1b was first freed of agglomerates larger than 150 µm that are formed by adhering material in the rotary tube or spray tower by means of coarse screening. Subsequently, by means of air classification, the desired grain band was established by removal of fine particles.

The final white spherical support material had a D10 of 36 µm, a D50 of 70 µm, a D90 of 113 µm, a fines fraction below 25 µm of less than 2.5% by volume, a coarse fraction larger than 150 µm of less than 0.1% by volume, an average sphericity of greater than 0.8 and an average symmetry of greater than 0.85. Sphericity and symmetry were determined by means of dynamic image evaluation (Retsch HORIBA Camsizer X2) as the variance of the 2D-projected particle surface from an ideal circle, with a value of 1 corresponding to a perfect sphere or circle in 2D projection. The BET was 140 m²/g, the pore volume was 0.34 mL/g, and the pore diameter was 8.1 nm. The carrier material was amorphous and the individual components were distributed randomly; 86.8% by weight of SiO₂, 5.8% by weight of MgO and 7.4% by weight of Al₂O₃ were present.

The yield in steps 1a to 1c was more than 80%.

### Comparative Example 1a - Support Production With Reduced Dwell Time and Catalyst Synthesis, and Batch Testing

The support was produced analogously to examples 1a to 1c on a 10 kg scale, except that the dwell time in the calcination was reduced to 15 minutes, resulting in a nitrate content in the support of 10 000 ppm.

200 mg of the resultant support material was suspended in 20 g of demineralized water in a pressure vessel and heated to 180° C. for 1 h. After cooling, the loss of magnesium and silicon was measured as a measure of hydrolysis stability. The support, by comparison with the support material from examples 1a to 1c, has four times the loss of magnesium and silicon, as a result of which the support material has lower mechanical stability.

The catalyst was produced analogously to example 2a on a 1 kg scale. The elevated nitrate content in the support resulted in an increase in the amount of wash water, as a result of which somewhat less gold was impregnated in the final catalyst. The final gold content was 0.78% by weight.

A steel autoclave with magnetic stirrer was initially charged with 384 mg of catalyst, which was suspended with a mixture of methacrolein (1.20 g) and methanol (9.48 g). The methanolic solution contained 50 ppm of Tempol as stabilizer. The steel autoclave was closed, 7% by volume of air was injected to 30 bar, and the mixture was stirred at 60 degrees for 2 hours. The mixture was cooled down to -10° C., the autoclave was cautiously degassed, and the suspension was filtered and analyzed by GC. The conversion of methacrolein was about 61%; the selectivity for MMA was 89%.

The example shows that shortening of the dwell time in the support calcination and the accordingly elevated nitrate contents lead to problems in catalyst production and in the synthesis of MMA from methacrolein and methanol. As well as the more marked hydrolytic instability which is critical for long-term use, it is also possible to find only a lower catalyst performance in short-term production.

### Example 2a - Catalyst Manufacture

An enamel tank with a propeller stirrer was initially charged with 167 kg of demineralized water, and 50 kg of the support material from example 1c was added. The steps that follow were conducted under isothermal conditions by means of steam heating of the reactor. Directly thereafter, a solution of 611 g of aluminum nitrate nonahydrate in 10 kg of demineralized water was added. The suspension was heated to 90° C. and then aged for 15 minutes. 2845 g of cobalt nitrate hexahydrate was dissolved in 20 kg of demineralized water and, on conclusion of the aging, metered in over the course of 10 minutes and reacted with the support material for 30 minutes.

In parallel, 12.4 L of an NaOH solution was prepared such that the ratio of hydroxide ions to auric acid was 4.75. The NaOH solution was added over the course of 10 minutes, in the course of which the suspension darkened in color.

After addition of the NaOH solution, 1250 g of an auric acid solution (gold content 41%) in 20 kg of demineralized water was diluted and added to the reaction suspension over the course of 10 minutes and stirred for a further 30 minutes.

The reaction suspension was cooled down to 40° C. after the reaction and pumped into a centrifuge with a filter cloth, with recycling of the filtrate until a sufficient filtercake had been built up. The filtercake was washed with demineralized water until the filtrate had a conductivity below 100 µS/cm, followed by dewatering for 30 minutes. Thereafter, the filtercake had a residual moisture content of nearly 30% by weight. The filtrates were first pumped through a selective ion exchanger in order to remove residual cobalt, and then residual gold was absorbed on activated carbon. The recovery rate of the two metals after the reaction was greater than 99.5%, which was determined by ICP analysis.

Directly after conclusion of the dewatering, the filtercake was dried in a paddle drier at 105° C. down to a residual moisture content of 2%. The drying process in the paddle drier was conducted batchwise within 8 hours with addition of a drying gas - nitrogen in this case.

Directly after the drying, the dried material was fed continuously into the rotary tube described in example 1b, which was operated at 450° C. under air. The dwell time was adjusted to 30 minutes.

The final catalyst had a loading of 0.91% by weight of gold, 1.10% by weight of cobalt, 2.7% by weight of magnesium, a BET of 236 m²/g, a pore volume of 0.38 mL/g and a pore diameter of 4.1 nm.

### Example 2b - Catalyst Testing in a Continuous Direct Oxidative Esterification

In a stainless steel pressure vessel equipped with an EKATO Phasejet and an EKATO Combijet stirrer unit, 1 kg of the catalyst from example 1c was dispersed in methanol/water (95/5), establishing a solids concentration in the suspension of 9%. The suspension was pressurized under nitrogen to 5 bar absolute at a temperature of 80° C. Methacrolein and methanol were metered in continuously in a molar ratio of 1:4 in the feed, and a stabilizer content of TEMPOL of 100 ppm. The reactor was simultaneously gassed with oxygen, such that the oxygen concentration in the reactor offgas was adjusted to 4% by volume (explosion limit at 7.8% by volume of oxygen). The feed rate and hence dwell time were adjusted such that the catalyst space velocity was 10 mol of methacrolein/kg of catalyst x hr. The pH of the reaction was kept constant at 7 by adding a solution of 4% NaOH, 5.5% H₂O and 90.5% methanol. The reaction was operated continuously with this setup for 2000 hours. The averaged conversion of methacrolein was about 80%; the selectivity for MMA was 94.5%. Conversion and selectivity were determined by means of GC-FID. MMA selectivity was unchanged within the scope of measurement accuracy (+/- 0.5%) over the 2000 hours of operation; conversion varied from initial values of 82% to 79% in the first nearly 500 hours and remained stable at that level for the rest of the operating time.

### Example 2c - Variation of Catalyst Drying Time and Testing

The catalyst was produced on a 1 kg scale analogously to example 2a on the support from example 1c, except that the drying time was extended this time from 8 to 20 hours. The residual moisture content after drying was 1%.

The catalyst was tested analogously to example 2b in a smaller test apparatus suitable for the use of 100 g of catalyst. After operation for 1000 hours, the averaged conversion of methacrolein was 75% and the selectivity for MMA was 94.5%.

Example 2c shows that, compared to example 2b, with prolonged drying time, a significant influence on conversion and hence activity was found in continuous sustained operation. Comparative example 2a thus shows a greater loss of initial activity, with no further deactivation detected after an initial decline in conversion.

### Example 2d - Variation of Catalyst Drying Time and Testing

The catalyst was produced on a 1 kg scale analogously to example 2a on the support from example 1c, except that the drying time was extended this time from 8 to 40 hours. The residual moisture content after drying was 0.8%.

The catalyst was tested analogously to example 2b in a smaller test apparatus suitable for the use of 100 g of catalyst. After operation for 1000 hours, the averaged conversion of methacrolein was 70% and the selectivity for MMA was 94.5%.

Example 2d shows that, compared to example 2b, with prolonged drying time, a significant influence on conversion and hence activity was found in continuous sustained operation. Comparative example 2a thus shows a greater loss of initial activity, with only minimal deactivation detected after an initial decline in conversion.

### Comparative Example 2a - Variation of Catalyst Drying Time and Testing

The catalyst was produced on a 1 kg scale analogously to example 2a on the support from example 1c, except that the drying time was extended this time from 8 to 70 hours. The residual moisture content after drying was 0.8%.

The catalyst was tested analogously to example 2b in a smaller test apparatus suitable for the use of 100 g of catalyst. After operation for 1000 hours, the averaged conversion of methacrolein was 64% and the selectivity for MMA was 92.5%. Conversion was unstable and dropped by more than 5% over the period.

Comparative example 2a shows that, compared to example 2b, with prolonged drying time, a significant influence on the conversion and hence activity was found in continuous sustained operation. The catalyst with a drying time of 70 hours, as well as the initial loss of activity, also showed a continuous decline in activity and conversion during further operation.

### Comparative Example 2b - Support Without Classification, Catalyst Synthesis and Testing

The support was produced on a 10 kg scale according to example 1a and 1b, except dispensing with the classification step. The fines fraction below 25 µm was larger this time at 10% by volume. The catalyst was produced on this unclassified support material on a 1 kg scale according to example 2a. The testing was effected according to example 2c, but this had to be stopped less than 24 hours after it had started since the fines fraction blocked the sintered metal filter of the reactor and only 60% of the initial discharge could be achieved. Up to that point, an averaged methacrolein conversion of 85% was achieved at an MMA selectivity of 94.5%.

The blockage could not be sufficiently remedied by purging with reaction mixture and nitrogen.

Comparative example 2b shows that the reaction without removal of fines proceeds chemically without losses in conversion and selectivity, but the effect of the fines fraction on the reaction equipment does not permit continuous sustained operation.

### Comparative Example 2c - Catalyst Synthesis Without Formation of a Protective Shell Layer

The catalyst was produced on a 1 kg scale analogously to example 2a on the support from example 1c, except that there was no addition of aluminum nitrate this time.

The catalyst was tested analogously to example 2b in a smaller test apparatus suitable for the use of 100 g of catalyst. After operation for 1000 hours, the averaged conversion of methacrolein was 75% and the selectivity for MMA was 94.3%. The conversion was at first unstable and fell by nearly 5%, but thereafter remained stable. MMA selectivity was unaffected.

Comparative example 2c shows that, compared to example 2b, in the absence of addition of aluminum salt and hence of a protective (outer) shell, a functioning catalyst is obtained, but a drop in catalyst activity is observed as a result of abrasion of gold and cobalt in outer layers. After abrasion of the outer active components, activity remains constant, but at a lower level. The loss of gold was quantified 0.10% absolute; the loss of cobalt in the same period was 0.15% absolute.

### Example 3a - Sieving and Batch Testing of Catalyst

1 kg of the catalyst prepared in comparative example 2b was sieved by means of various sieves and an agitating tower. For avoidance of blockage of the sieves, the individual sieves were cleaned periodically by compressed air. The sieving was effected into 6 fractions:

| Fraction [no., µm] | 1 | 2 | 3 | 4 | 5 | 6 |
|---|---|---|---|---|---|---|
| | > 100 | 80 - 100 | 63 - 80 | 40-63 | 20-40 | <20 |
| Sieve [µm] | 100 | 80 | 63 | 40 | 20 | remainder |

The individual fractions were analyzed by means of laser diffraction and ICP with regard to their grain size distribution and gold and cobalt contents:

| | | | | | | |
|---|---|---|---|---|---|---|
| Fraction | 1 | 2 | 3 | 4 | 5 | 6 |
| D50 [µm] | 107 | 88 | 71 | 54 | 36 | 15 |
| Au [%] | 0.72 | 0.81 | 1.00 | 1.20 | 1.73 | 2.48 |
| Co [%] | 0.89 | 0.96 | 1.12 | 1.26 | 1.59 | 2.14 |
| Au / Co | 0.81 | 0.84 | 0.89 | 0.94 | 1.09 | 1.16 |

In the context of this invention, grain size distribution was determined by means of ISO 13320:2020 Particle size analysis - Laser diffraction methods.

It is readily apparent that the gold and cobalt content rises as the particle diameter falls, with a greater than proportional rise in the case of gold. It is thus of particularly high interest for the fines fraction to be removed at the early stage of the support because, firstly, the catalyst based on the fines fraction adversely affects the filtration equipment and hence the reaction equipment and, secondly, this unwanted catalyst fines content takes up a disproportionate amount of gold and cobalt. This can lower precious metal costs. In addition, the smallest particles must be removed in order to prevent the cobalt present therein from ending up in wastewater, where these particles are toxic to man and the environment on account of the cobalt.

A steel autoclave with magnetic stirrer was initially charged with 384 mg of catalyst of the respective fraction from, which was suspended with a mixture of methacrolein (1.20 g) and methanol (9.48 g). The methanolic solution contained 50 ppm of Tempol as stabilizer. The steel autoclave was closed, 7% by volume of air was injected to 30 bar, and the mixture was stirred at 60 degrees for 2 hours. The mixture was cooled down to -10° C., the autoclave was cautiously degassed, and the suspension was filtered and analyzed by GC. In order to detect the selectivity for methyl isobutyrate, 1% by weight of sodium formate was added to the mixture, which serves as reduction equivalent.

| | | | | | | |
|---|---|---|---|---|---|---|
| Fraction | 1 | 2 | 3 | 4 | 5 | 6 |
| MAL conversion | 65.1% | 70.7% | 74.3% | 80.3% | 86.3% | 92.1% |
| Methyl isobutyrate selectivity | 0.36% | 0.39% | 0.44% | 0.51% | 0.55% | 0.59% |

In line with the ICP results, a higher activity is found in the case of smaller particles, and also a higher selectivity for methyl isobutyrate; this should be kept as low as possible in the final on-spec product in order to be able to use the MMA for optical applications inter alia. The maximum methyl isobutyrate content must ultimately be significantly below 1000 ppm. Depletion as a result of the process through, for example, rectification, distillation, extraction and hydrolysis is very difficult and requires such high capital costs and running costs that a process is of no economic and/or industrial interest. This again shows that a support without appropriate classification is unsuitable for catalyst production and reaction.

### Example 4a - Alternative Catalyst Production With PVP & Sodium Citrate (Precolloid Formation)

An enamel tank with a propeller stirrer was initially charged with 16.7 kg of demineralized water, and 5 kg of the support material from example 1c was added. The steps that follow were conducted under isothermal conditions by means of steam heating of the reactor. Directly thereafter, a solution of 61.1 g of aluminum nitrate nonahydrate in 1 kg of demineralized water was added. The suspension was heated to 90° C. and then aged for 15 minutes. In parallel, 284.5 g of cobalt nitrate hexahydrate was dissolved in 2 kg of demineralized water and, on conclusion of the aging, metered in over the course of 10 minutes and reacted with the support material for 30 minutes.

In parallel, 1.24 L of an NaOH solution was prepared such that the ratio of hydroxide ions to auric acid is 4.75. The NaOH solution was added over the course of 10 minutes, in the course of which the suspension darkened in color.

In a second enamel tank with a propeller stirrer, 62.5 g of auric acid was diluted in 2 kg of demineralized water, and 65 g of polyvinylpyrrolidone (average molecular weight 8000 to 10 000 g/mol) was added. After stirring briefly, 62.5 g of sodium citrate was added and the mixture was heated to 70° C., which formed a purple to black colloid solution within 0.5 hour.

The colloid solution was pumped into the support suspension, and the resultant mixture was cooled passively to room temperature, which was followed by a period of further stirring for 10 hours.

The suspension was then washed, centrifuged, dried and calcined analogously to example 2a. The calcination additionally oxidatively removed the polyvinylpyrrolidones from the gold nanoparticles.

As a result of the polyvinylpyrrolidone stabilization, complete adsorption of the gold nanoparticles in the filtrate & wash solution from the catalyst system on activated carbon for recovery of the precious metal took about 24 hours, which is much longer than in the case of the synthesis method without polyvinylpyrrolidones.

The final catalyst had a loading of 0.48% by weight of gold and 1.09% by weight of cobalt.

### Example 4b - Alternative Catalyst Production With PVP & Sodium Citrate (Precolloid Formation)

The synthesis was conducted analogously to example 4a, except that 195 g of copper nitrate and 156 g of lanthanum nitrate were used in place of cobalt nitrate and there was no addition of NaOH solution. In addition, the catalyst, after the calcination, was converted to a reductive form by means of addition of hydrogen at 100° C. for 1 hour.

The final catalyst had a loading of 0.48% by weight of gold, 1.01% by weight of copper and 0.96% by weight of lanthanum. It was thus found that the deposition of lanthanum is complete at 99% of theory, but only 58% of the theoretical deposition was possible in the case of copper. Since copper nitrate is very toxic to water organisms, a complex recovery of the copper must take place here analogously to the removal of cobalt.

### Example 4c - Catalyst Testing in a Continuous Direct Oxidative Esterification

The catalyst from example 4a was tested analogously to example 2b in a smaller test apparatus suitable for the use of 100 g of catalyst. After operation for 1000 hours, the averaged conversion of methacrolein was 45.8% and the selectivity for MMA was 91.0%.

### Example 4d - Catalyst Testing in a Continuous Direct Oxidative Esterification

The catalyst from example 4a was tested analogously to example 2b in a smaller test apparatus suitable for the use of 100 g of catalyst. After operation for 1000 hours, the averaged conversion of methacrolein was 47.3% and the selectivity for MMA was 91.5%.

1. A process for producing a catalyst for an oxidative esterification, the process comprising:
(a) producing an oxidic support containing at least one or more oxides of silicon, of aluminum, of one or more alkaline earth metals, of titanium, of zirconium, of hafnium, of vanadium, of niobium, of tantalum, of yttrium, and/or of lanthanum, the producing comprising
(a) (i) reacting one or more compounds selected from the group consisting of silicon compounds, aluminum compounds, alkaline earth metal compounds, titanium compounds, zirconium compounds, hafnium compounds, vanadium compounds, niobium compounds, tantalum compounds, yttrium compounds, and lanthanum compounds, at a temperature T₁ < 100° C. to obtain a suspension,
(a) (ii) spray-drying the suspension at a temperature T₂ > 110° C., to obtain a solid-state material having 0.1 % to 20% by weight of water and 0.1% to 35% by weight of anions of one or more Brønsted acids,
(a) (iii) calcining the solid-state material at a temperature T₃ between 300 to 800° C. to obtain the oxidic support of a second solid-state material having 0.01 % to 5% by weight of water and 0.01% to 0.5% by weight of the anions of one or more Brønsted acids, and
(a) (iv) optionally, subjecting the oxidic support to classifying, and
(b) converting the oxidic support from a to a catalyst, the converting comprising
(b) (i) reacting the oxidic support from (a) with a water-soluble precious metal salt,
(b) (ii) simultaneously or subsequently adding a further soluble metal salt, to obtain an impregnated support in a mother liquor,
(b) (iii) removing the impregnated support from the mother liquor and then washing the impregnated support, wherein the impregnated support after washing contains 1.0% to 50% by weight of water.
(b) (iv) drying the impregnated support for 0.1 to 40 h at a temperature T₄ between 30 and 250° C. to obtain a dried impregnated support having 0.1% to 10% by weight of water, and
(b) (v) calcining the dried impregnated support from (iv) at a temperature T₅ between 250 and 700° C. and for a residence time between 0.1 and 5 h to obtain the catalyst having a BET surface area of 100 to 300 m²/g with a pore volume of 0.2 to 2.0 m/g and a pore diameter of 3 to 12 nm.

2. The process as claimed in claim 1,
wherein (a) (i), (b) (i), and (b) (ii) are conducted batchwise, and wherein (a) (ii) and (a) (iii) are conducted continuously or semicontinuously.

3. The process as claimed in claim 1,
wherein in (a) (iv), the second solid-state material from is treated in such a way that a proportion of particles having a diameter of less than 20 µm is reduced.

4. The process as claimed in
claim 1, wherein, in or after (b) (ii), a basic aqueous solution is additionally added.

5. The process as claimed in
claim 1, wherein the mother liquor removed in (b) (iii) is worked up in such a way that remaining precious metal salts and other metal salts are recovered.

6. The process as claimed in
claim 1, wherein the drying in (b) (iv) is effected at an absolute pressure between 0.01 and 5 bar and/or in the presence of an inert drying gas.

7. The process as claimed in
claim 1, wherein the calcination in (a) (iii) and optionally, the calcination in (b) (v), is effected batchwise.

8. The process as claimed in
claim 1, wherein the calcination in (a) (iii) and optionally, the calcination in (b) (v), is effected continuously or semicontinuously in a rotary tube.

9. The process as claimed in
claim 1, wherein, in (b) (i), an aqueous suspension of the oxidic support from (a) is produced and is mixed with the water-soluble precious metal salt.

10. The process as claimed in
claim 1, wherein the oxidic support comprises silicon oxide, aluminum oxide, and at least one alkaline earth metal oxide.

11. The process as claimed in
claim 1, wherein the solid-state material after (a) (ii), the second solid-state material after (a) (iii), or the oxidic support after (a) (iv) is subjected to shaping in such a way that a shaped body having a diameter between 0.1 and 100 mm is obtained.

12. The process as claimed in
claim 1, wherein a time between spray-drying and calcination in (a) (ii) and (a) (iii) is not longer than 5 days.

13. The process as claimed in
claim 1, wherein a time between washing and calcination in (b) (iv) and (b) (v) is not longer than 4 days.

14. The process as claimed in
claim 1, wherein, before, during, or after the reaction of the oxidic support in (b) (i) and (b) (ii), a water-soluble Bronsted or Lewis acid is added.

15. A method, comprising:
continuously preparing a carboxylic acid by reaction of an aldehyde and an alcohol in the presence of an oxygenous gas in a liquid phase, wherein a catalyst is suspended heterogeneously in a reaction matrix,
wherein the catalyst is produced by the process according to claim 1.

16. The method as claimed in claim 15,
wherein the reaction is effected at a temperature between 20 and 120° C., a pH between 5.5 and 9, and a pressure between 1 and 20 bar, and
wherein a reaction solution of the reaction contains between 2% and 10% by weight of water.

17. A method, comprising:
continuously preparing a carboxylic acid by reaction of an aldehyde and an alcohol in the presence of an oxygenous gas in a liquid phase,
wherein the reaction comprises a catalyst the form of a fixed bed,
wherein the catalyst is produced by the process according to claim 11.

18. A catalyst, producible by the process as claimed in claim 1.

19. The process as claimed in claim 14, wherein the water-soluble Bronsted or Lewis acid is an aqueous solution of a metal salt having the +II or +III oxidation state.
